(19) United States
(12) Patent Application Publication
(10) Pub. No.: US 2024/0219215 A1
(43) Pub. Date: Jul. 4, 2024

(54) LIQUID MONITORING SYSTEM AND METHOD

(71) Applicant: REALMFIVE, Inc., Lincoln, NE (US)

(72) Inventors: Josh Behlen, Lincoln, NE (US); Mateusz Mittek, Lincoln, NE (US); Brant Burkey, Denton, NE (US); Steve R Tippery, Gretna, NE (US)

(73) Assignee: REALMFIVE, Inc., Lincoln, NE (US)

(21) Appl. No.: 18/244,119

(22) Filed: Sep. 8, 2023

Related U.S. Application Data
(63) Continuation-in-part of application No. 16/936,304, filed on Jul. 22, 2020, now Pat. No. 11,815,387.
(60) Provisional application No. 63/404,835, filed on Sep. 8, 2022.
(60) Provisional application No. 62/877,092, filed on Jul. 22, 2019.

(51) Int. Cl. G01F 1/66; G01F 23/292; G01F 23/80

(52) U.S. Cl. CPC G01F 1/666; G01F 23/292; G01F 23/808

(57) ABSTRACT

A liquid monitoring system may include a monitoring device configured to be positioned at a surface of a confined liquid body. The monitoring device may include a housing including walls defining a cavity. The monitoring device may include an internal sensing sub-system enclosed within the cavity. The internal sensing sub-system may include one or more liquid monitoring sensors configured to monitor a level, temperature, or quality of the confined liquid body. The liquid monitoring system may be configured to receive a set of liquid monitoring data from the one or more liquid monitoring sensors. The liquid monitoring system may be further configured to determine one or more liquid measurement values based on set of liquid monitoring data. The one or more liquid measurement values may include a liquid level measurement value, a temperature measurement value, or a liquid quality measurement value for the confined liquid body.

### CROSS-REFERENCE TO RELATED APPLICATION

The present application claims the benefit under 35 U.S.C. § 119(e) of U.S. Provisional Application Ser. No. 63/404,835, filed Sep. 8, 2022, and also constitutes a continuation-in-part (CIP) of U.S. Non-Provisional patent application Ser. No. 16/936,304, filed Jul. 22, 2020, which is a non-provisional application of U.S. Provisional Application Ser. No. 62/877,092, filed Jul. 22, 2019, whereby each of the above-listed patent applications are each incorporated herein by reference in their entirety.

### TECHNICAL FIELD

The present disclosure relates generally to liquid monitoring, and, more particularly, to a system and method for liquid monitoring using non-contact sensing.

### BACKGROUND

In a wide range of applications, from natural bodies of water, artificial reservoirs, volumes of liquid held in storage tanks, and the like, it may be desirable to monitor and/or manage various aspects of a liquid. Accordingly, liquid monitoring systems are sometimes used to obtain data regarding one or more characteristics of the liquid (e.g., liquid level or depth, temperature, quality, or the like). However, existing liquid monitoring solutions impose inherent limitations and challenges due to their reliance on direct-contact sensing, fixed mounting points, physical cables for power/data transmission, and onsite data access. These limitations underscore the critical need for a more innovative and efficient approach to liquid level measurement and tank monitoring. As such, it would be desirable to provide a system and method which cures one or more of the shortfalls of the previous approaches identified above.

### SUMMARY

A liquid monitoring system is disclosed, in accordance with one or more embodiments of the present disclosure. In embodiments, the liquid monitoring system includes a monitoring device configured to be positioned at a surface of a confined liquid body. In embodiments, the monitoring device includes a housing including one or more walls at least partially defining a cavity. In embodiments, the monitoring device includes an internal sensing sub-system. In embodiments, the cavity of the housing is configured to enclose the internal sensing sub-system. In embodiments, the internal sensing sub-system includes one or more liquid monitoring sensors configured to monitor at least one of a level, a temperature, or a quality of the confined liquid body. In embodiments, the liquid monitoring system further includes one or more processors communicatively coupled to the one or more liquid monitoring sensors. In embodiments, the one or more processors are configured to execute a set of program instructions in a memory. In embodiments, the set of program instructions are configured to cause the one or more processors to receive a set of liquid monitoring data from the one or more liquid monitoring sensors. In embodiments, the set of liquid monitoring data includes at least one of liquid level data, liquid temperature data, or liquid quality data for the confined liquid body. In embodiments, the set of program instructions are configured to cause the one or more processors to determine one or more liquid measurement values based on set of liquid monitoring data. In embodiments, the determined one or more liquid measurement values include at least one of a liquid level measurement value, a temperature measurement value, or a liquid quality measurement value for the confined liquid body.

A liquid monitoring system is disclosed, in accordance with one or more embodiments of the present disclosure. In embodiments, the one or more processors are configured to execute a set of program instructions in a memory. In embodiments, the set of program instructions are configured to cause the one or more processors to receive a set of liquid monitoring data from the one or more liquid monitoring sensors. In embodiments, the set of liquid monitoring data includes at least one of liquid level data, liquid temperature data, or liquid quality data for the confined liquid body. In embodiments, the one or more liquid monitoring sensors have no direct contact with the confined liquid body. In embodiments, the set of program instructions are configured to cause the one or more processors to determine one or more liquid measurement values based on set of liquid monitoring data. In embodiments, the determined one or more liquid measurement values include at least one of a liquid level measurement value, a temperature measurement value, or a liquid quality measurement value for the confined liquid body.

A method is disclosed, in accordance with one or more embodiments of the present disclosure. In embodiments, the method includes receiving a set of liquid monitoring data from one or more liquid monitoring sensors. In embodiments, the one or more liquid monitoring sensors have no direct contact with a confined liquid body. In embodiments, the set of liquid monitoring data includes at least one of liquid level data, liquid temperature data, or liquid quality data. In embodiments, the method includes determining one or more liquid measurement values based on set of liquid monitoring data. In embodiments, the determined one or more liquid measurement values include at least one of a liquid level measurement value, a temperature measurement value, or a liquid quality measurement value for the confined liquid body.

It is to be understood that both the foregoing general description and the following detailed description are exemplary and explanatory only and are not necessarily restrictive of the invention as claimed. The accompanying drawings, which are incorporated in and constitute a part of the specification, illustrative embodiments of the invention and together with the general description, serve to explain the principles of the invention.

### BRIEF DESCRIPTION OF DRAWINGS

The numerous advantages of the disclosure may be better understood by those skilled in the art by reference to the accompanying figures in which:

FIG. 1A is a simplified block diagram of a liquid monitoring system, in accordance with one or more embodiments of the present disclosure.

FIG. 1B is a simplified block diagram of a liquid monitoring system, in accordance with one or more embodiments of the present disclosure.

FIG. 2A is a simplified block diagram of a monitoring device of the liquid monitoring system, in accordance with one or more embodiments of the present disclosure.

FIG. 2B is a schematic view of the monitoring device of the liquid monitoring system, in accordance with one or more embodiments of the present disclosure.

FIG. 2C is a cross-sectional view of the monitoring device of the liquid monitoring system, in accordance with one or more embodiments of the present disclosure.

FIG. 3A is a perspective view of a liquid monitoring system, in accordance with one or more embodiments of the present disclosure.

FIG. 3B is a perspective view of a liquid monitoring system including an external sensor, in accordance with one or more embodiments of the present disclosure.

FIG. 3C is a perspective view of a liquid monitoring system including a Radio Frequency Identification (RFID) reading device, in accordance with one or more embodiments of the present disclosure.

FIG. 3D is a perspective view of a liquid monitoring system utilizing a real-time kinetic (RTK) technique, in accordance with one or more embodiments of the present disclosure.

FIG. 4 is a process flow diagram illustrating the steps of a method of liquid monitoring, in accordance with one or more embodiments of the present disclosure.

FIG. 5A is a conceptual view of a user interface of the liquid monitoring system, in accordance with one or more embodiments of the present disclosure.

FIG. 5B is a conceptual view of a display of a mobile device in communication with the platform server of the liquid monitoring system, in accordance with one or more embodiments of the present disclosure.

FIG. 5C is a top view of a map including a position of a liquid monitoring system, in accordance with one or more embodiments of the present disclosure.

FIG. 5D is a graphical representation of a liquid level of a liquid monitoring system, in accordance with one or more embodiments of the present disclosure.

FIG. 5E is a conceptual view of a liquid level threshold setting of a liquid monitoring system, in accordance with one or more embodiments of the present disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Smart tank monitoring systems or smart tank solutions are advanced technology platforms designed to monitor and manage various aspects of liquid storage tanks, such as fuel tanks, water tanks, chemical tanks, and more. These systems leverage a combination of sensors, wireless communication, data analytics, and software applications to provide real-time data and insights about the status, levels, and conditions of the stored liquid. Smart tank systems find applications in various industries, including agriculture, oil and gas, water treatment, chemicals, logistics, and more. They offer advantages such as improved operational efficiency, reduced maintenance costs, increased safety, and the ability to make data-driven decisions, making them valuable tools for organizations seeking to optimize their liquid storage and management processes. In an era where precision, sustainability, and resource optimization are paramount, smart tank monitoring systems have emerged as indispensable tools for ensuring the seamless operation and intelligent management of liquid assets.

Existing solutions have long been tethered to conventional methodologies that impose inherent limitations and challenges. The primary issues plaguing these traditional systems stem from their reliance on direct-contact sensing, fixed mounting points, physical cables for power/data transmission, and onsite data access.

For example, some existing solutions takes advantage of the characteristics of pressure and utilize a tube external from the liquid body, where the tube has an inlet near the bottom and top of the confined space to allow an operator external from the liquid body to see the physical level. However, such solutions have a number of disadvantages including, but not limited to, the tube must be kept in clean condition to operate properly, the fixed installation correlates to a lengthy and complex installation, and the level must be read onsite.

By way of another example, some existing solutions use pressure to determine depth in a liquid body and a tube internal to the liquid body is utilized, where the tube extends from the top to near the bottom of the confined space and a force transducer lives in the top of that tube. As the height of the liquid body changes the transducer relates the pressure change in the tube to a specific level. However, such solutions have a number of disadvantages including, but not limited to, the tube must be kept in clean condition to operate properly (i.e., depending on the composition of the liquid body material buildup could occur on the inside of the tube which may skew the measurements), the fixed installation correlates to a lengthy and complex installation, and a difference in liquid body composition means the algorithm to translate pressure to depth must be modified.

By way of a further example, additional existing solutions use a float method to determine depth in a liquid body. This solution uses a float internal to the liquid body in series with mechanical pulleys, cables, and counterweights, which allows an operator external from the liquid body to see the physical level. However, such solutions have a number of disadvantages. For example, such systems must be calibrated after initial installation, a difference in liquid body composition means the system must be re-calibrated, the fixed installation correlates to a lengthy and complex installation, mechanical features wear over time and must be serviced, and the liquid level must be read onsite.

As such, there is a need for a liquid monitoring system and method, which cures one or more of the shortfalls of previous approaches identified above.

Embodiments of the present disclosure are directed to a liquid monitoring system and method to determine one or more characteristics of liquid within a confined space. For example, the system and method may utilize non-contact sensing via a monitoring device including one or more sensors enclosed within a protective housing, where the one or more sensors measure and/or collect data for one or more characteristics of the liquid within the confined space. For instance, the one or more sensors may utilize non-contact sensing techniques such as, but are not limited to, sonar, lidar, acceleration, real-time kinetic (RTK) positioning techniques, and the like to measure liquid level (or depth). Additionally, the one or more sensors may utilize non-contact sensing techniques such as, but are not limited to, conductivity, infrared radiation, and the like to measure liquid temperature. Further, the one or more sensors may utilize non-contact sensing techniques such as, but are not limited to, conductivity-based techniques, and the like to assess the cleanliness of the liquid body. As such, the system and method may enable users to seamlessly track and manage their liquid assets with a significant level of accuracy and efficiency.

It is contemplated herein that the non-contact liquid level determination system and method of the present disclosure, offer several distinct advantages. For example, installation is quick and intuitive, as the monitoring device can simply be placed onto the liquid surface, eliminating the need for complex and time-consuming installation procedures. By way of another example, the system and method may accurately measure levels of any liquid body, irrespective of the liquid's composition, without necessitating alterations to the device itself, making it highly adaptable across various industries and applications. Moreover, the device's robust design enables it to operate effectively in dirty environments, ensuring its reliability even in adverse conditions. Finally, the system and method allows for seamless integration with cloud-based communication technologies to enable real-time reporting of measurements to the cloud, allowing for remote monitoring of liquid levels from anywhere in the world, providing enhanced convenience and accessibility to users. As such, this approach may eliminate the need for direct contact, fixed mounting points, and physical cables.

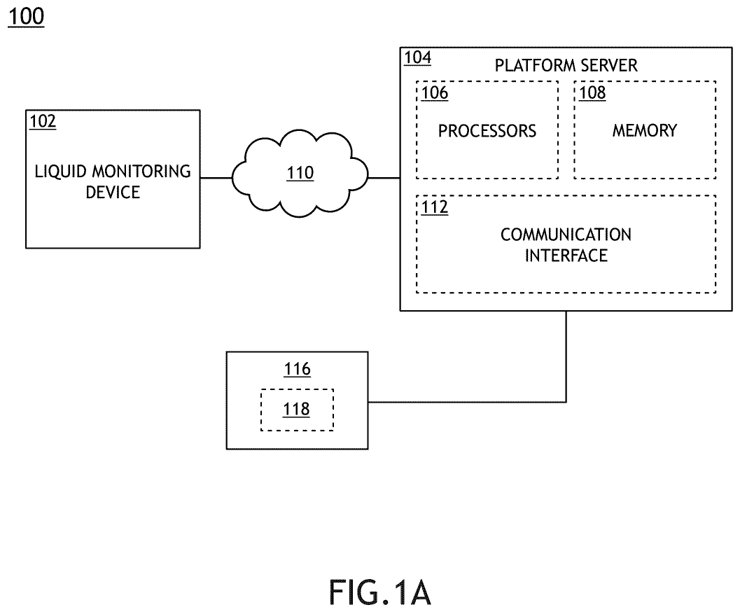

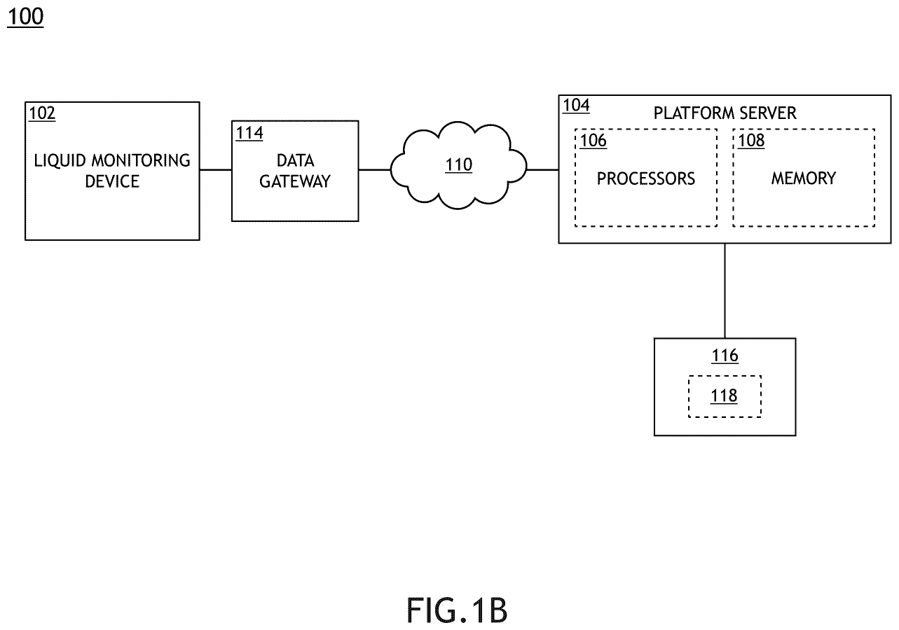

Referring generally to FIGS. 1A-5E, a system and method for liquid level monitoring are disclosed, in accordance with one or more embodiments of the present disclosure.

FIGS. 1A-1B illustrate simplified block diagrams of a liquid monitoring system 100, in accordance with one or more embodiments of the present disclosure.

In embodiments, the liquid monitoring system 100 includes a monitoring device 102. The monitoring device 102 may be positioned within a confined liquid body to measure and/or collect data regarding one or more characteristics of the liquid. For example, as will be discussed further herein, the monitoring device 102 may be configured to utilize non-contact sensing to measure and/or collect data regarding one or more characteristics of the liquid, where one or more liquid monitoring sensors 101 do not make contact with the liquid of the confined liquid body. The one or more characteristics of the liquid measured by the one or more liquid monitoring sensors 101 may include, but are not limited to, liquid level (e.g., vertical depth or position of the device on a surface of the liquid within the confined space), temperatures, cleanliness (or quality), and the like.

As will be described in further detail herein, it is contemplated herein that the liquid monitoring system 100 of the present disclosure may be implemented in a wide variety of contexts, and may be used to measure characteristics of any liquid. For example, monitoring device 102 may be configured to measure material characteristics of water, oil, liquid fertilizer, and the like. For instance, the liquid monitoring system 100 of the present disclosure may measure characteristics of water within a water tank for livestock, a run-off pond, irrigation, swimming pool, hot tub, or the like. In this regard, although liquid monitoring system 100 may be described as being implemented in an agricultural context, this should not be interpreted as a limitation on the scope of the present disclosure as it is contemplated that the liquid monitoring system 100 of the present disclosure may be implemented in any number of contexts.

Referring generally to FIGS. 1A-1B, in embodiments, the liquid monitoring system 100 includes one or more platform servers 104. The one or more platform servers 104 may include one or more processors 106 configured to execute program instructions maintained on a memory medium 108. In this regard, the one or more processors 106 of the one or more platform servers 104 may execute any of the various process steps described throughout the present disclosure. It is contemplated herein that the one or more platform servers 104 may include a remote server configured to carry out one or more of the steps of the present disclosure. In embodiments, the one or more platform servers 104 may include a cloud-based computing platform including, but not limited to, Amazon Web Services (AWS).

In embodiments, as shown in FIG. 1A, the data collected by the monitoring device 102 may be directly transmitted to the one or more platform servers 104 via the network 110. For example, the one or more platform servers 104 and/or the monitoring device 102 may include a network interface device 112 and/or communication circuitry suitable for interfacing with the network 110. The communication circuitry 112 may include any network interface circuitry or network interface device suitable for interfacing with network 110. For example, the communication circuitry 112 may include a wireless-based interface device employing GSM, GPRS, CDMA, EV-DO, EDGE, WiMAX, 3G, 4G, 4G LTE, 5G, Wi-Fi protocols, RF, LoRa, and the like.

In embodiments, as shown in FIG. 1B, the data collected by the monitoring device 102 may be transmitted to the one or more platform servers 104 via a data gateway 114. The data gateway 114 may be configured to receive data from the monitoring device 102 and transmit the received data to the one or more platform servers 104 via network 110. The data gateway 114 may be configured to transmit data to the one or more platform servers 104 via network 110 using any type of wireless communication technique known in the art including, but not limited to, radio frequency (RF) protocols, Bluetooth protocols, GSM, GPRS, DCMA, EV-DO, EDGE, WIMAX, 4G, 4G LTE, 5G, 6G, WiFi protocols, RF, LoRa, and the like. In this regard, the data gateway 114 may include any network interface known in the art configured to communicatively couple the data gateway 114 to the network 110.

In embodiments, the data gateway 114 may be positioned on land. For example, the data gateway 114 may be installed at a location adjacent to a liquid body. By way of another example, the data gateway 114 may be installed at a location nonadjacent to the liquid body.

In embodiments, the liquid monitoring system 100 may further include or be coupled to a user device 116 communicatively coupled to the one or more platform servers 104. In embodiments, the user device 116 includes a display 118 used to display data of the liquid monitoring system 100 to a user. The display 118 of the user device 116 may include any display known in the art. For example, the display may include, but is not limited to, a liquid crystal display (LCD), an organic light-emitting diode (OLED) based display, or a CRT display. Those skilled in the art should recognize that any display 118 device capable of integration with a user device 116 is suitable for implementation in the present disclosure. In embodiments, a user may input selections and/or instructions responsive to data displayed to the user via the user device 116.

In embodiments, the user device 116 may include, but is not limited to, one or more desktops, laptops, tablets, smartphones, smart watches, or the like. In embodiments, a user may use the user device 116 in order to view data collected by the monitoring device 120, generated by the one or more processors 106, or stored in memory 188. In embodiments, the user device 116 may be configured one or more input commands from a user via one or more user input devices 120, wherein the one or more input commands are configured to cause one or more processors to adjust one or more characteristics of liquid monitoring system 100.

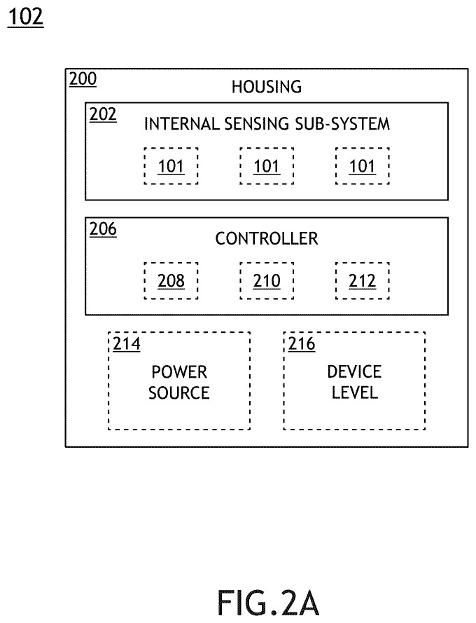

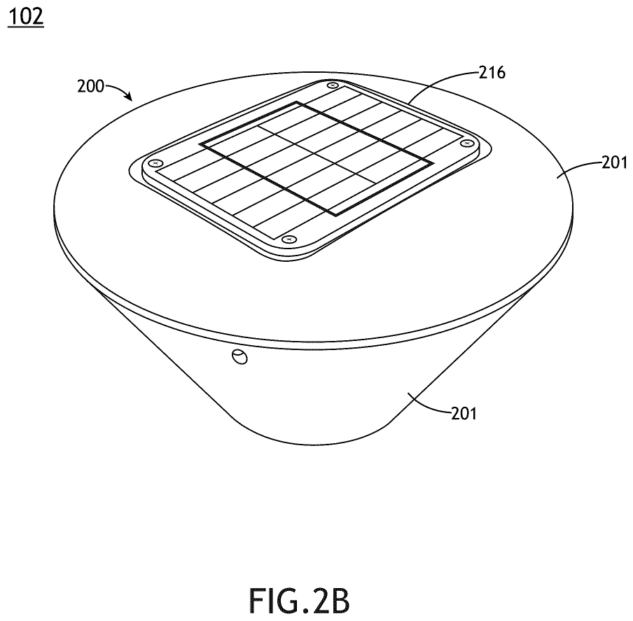

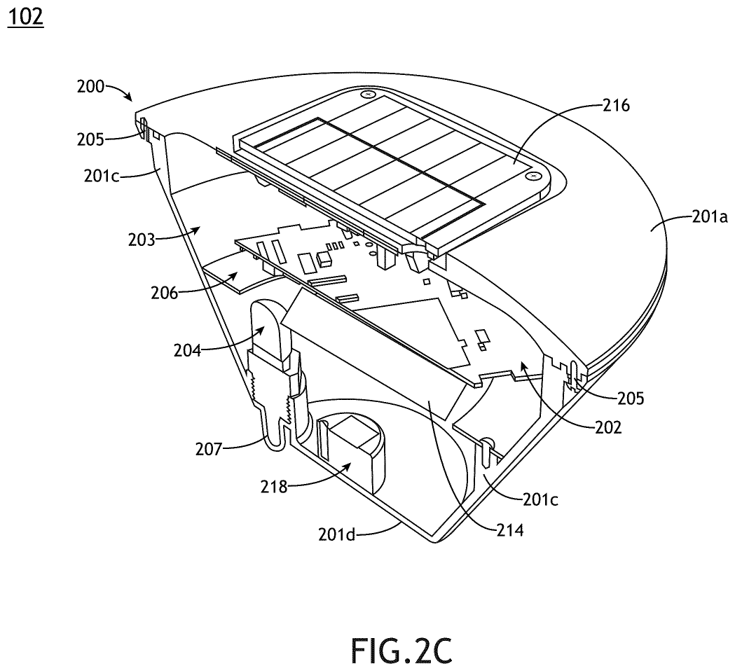

FIGS. 2A-2C illustrates the monitoring device 102 of the liquid monitoring system 100, in accordance with one or more embodiments of the present disclosure. In particular, FIG. 2A is a simplified block diagram of the monitoring device 102 of the liquid monitoring system 100 and FIGS. 2B-2C are schematic views of the monitoring device 102 of the liquid monitoring system, in accordance with one or more embodiments of the present disclosure. In embodiments, the monitoring device 102 includes, but is not limited to, a housing 200, an internal sensing sub-system 202, one or more liquid monitoring sensors 101, 204, a controller 206 including one or more processors 208, memory 210, and communication interface 212, a power source 214, one or more solar panels 216, a device level 218, or the like.

In embodiments, the monitoring device 102 includes a housing 200 (or casing). The housing 200 may include one or more walls 201 at least partially defining a cavity 203. For example, as shown in FIG. 2B, the housing 200 may include at least a top wall 201a, a rear (or bottom) wall 201b, a first side wall 201c, and a second side wall 201d. In this regard, the one or more walls 201 of the housing 200 may form a sealed casing around the one or more components within the cavity 203, such that the one or more components within the cavity 203 do not come in contact with the liquid.

The one or more walls 201 may be removably coupled together, such that components within the cavity 203 may be accessed. For example, the top wall 201a may be removably coupled to one of the first side wall 201c or the second side wall 201d via one or more fasteners 205. Although FIG. 2C depicts the top wall 201a being removably coupled to the first side wall 201c and the second side wall 201d via the one or more fasteners 205, it is contemplated herein that the one or more walls 201 may be removably coupled via any configuration or technique. For example, the one or more walls 201 may include an access port (or door). Further, although FIGS. 2B-2C depict a plurality of walls 201, it is contemplated herein that the housing 200 may be a single injection-molded housing 200 formed of a continuous single wall 201.

The one or more walls 201 of the housing 200 may be formed of any material suitable for making contact with a liquid body and protecting the one or more electronic components within the cavity 203. For example, the one or more walls 201 of the housing 200 may be formed of one or more thermoplastics, silicone, or the like. In one instance, the one or more components within the cavity 203 are protecting using potting (e.g., filling or embedding in the housing with a resinous material). In another instance, the one or more components within the cavity 203 are protected using encapsulation (e.g., creating a protective shell around the components without embedded them).

In embodiments, the monitoring device 102 includes an internal sensing sub-system 202 enclosed within the cavity 203 of the housing 200.

In embodiments, the internal sensing sub-system 202 includes one or more integrated circuits. The one or more integrated circuits 202 may include (or be coupled to) one or more liquid monitoring sensors 101 to measure one of level, temperature, or cleanliness (quality) of the liquid within the confined space.

The integrated circuit 202 may further include, but is not limited to, one or more microcontrollers, one or more microprocessors, one or more memory devices, and the like.

In embodiments, the monitoring device 102 may include one or more additional liquid monitoring sensors 204. For example, the one or more additional sensors 204 may be separate from the integrated circuit and arranged within the cavity 203 of the housing 200. For instance, as shown in FIG. 2C, the monitoring device 102 may include a temperature sensing probe 207, where the end of the probe is encased within the wall 201d such that liquid may surround the end of the probe to measure the temperature of the liquid without directly touching a surface of the temperature sensing probe 207.

Although FIG. 2C depicts the temperature sensing probe 207 being encased within the housing 200, it is contemplated herein the temperature sensing probe 207 may protrude through a wall of the housing 200.

The one or more liquid monitoring sensors 101 may include any suitable liquid monitoring sensor for measuring and/or collect data for one or more characteristics of the liquid. For example, the one or more liquid monitoring sensors 101 may include, but are not limited to, one or more ultrasonic sensors, one or more lidar sensors, one or more radar antennas, one or more acceleration sensors (e.g., accelerometers, gyroscopes, tilt switches, or the like), one or more temperature sensors (e.g., one or more infrared sensors, one or more conductivity sensors, or the like), one or more electrical conductivity sensors (e.g., contacting or inductive sensors), one or more pressure sensors, one or more humidity sensors, and the like.

The one or more additional liquid monitoring sensors 204 may include any suitable liquid monitoring sensor for measuring and/or collect data for one or more characteristics of the liquid. For example, the one or more liquid monitoring sensors 204 may include, but are not limited to, one or more ultrasonic sensors, one or more lidar sensors, one or more acceleration sensors (e.g., accelerometers, gyroscopes, tilt switches, or the like), one or more temperature sensors (e.g., one or more infrared sensors, one or more conductivity sensors, or the like), one or more electrical conductivity sensors (e.g., contacting or inductive sensors), one or more pressure sensors, one or more humidity sensors, and the like.

In embodiments, the monitoring device 102 includes a controller 206 enclosed within the cavity 203 of the housing 200. The controller 206 includes one or more processors 208 configured to execute program instructions maintained on a memory medium 210. In this regard, the one or more processors 208 may execute any of the various process steps described throughout the present disclosure. The controller 206 may include a network interface device 212 and/or communication circuitry suitable for interfacing with the network 110. The communication circuitry 212 may include any network interface circuitry or network interface device suitable for interfacing with network 110. For example, the communication circuitry 212 may include a wireless-based interface device employing GSM, GPRS, CDMA, EV-DO, EDGE, WiMAX, 3G, 4G, 4G LTE, 5G, Wi-Fi protocols, RF, LoRa, and the like.

In embodiments, the monitoring device 102 includes the power source 214 configured to provide power to one or more components within the housing 200. For example, the power source 214 may be configured to power one of the internal sensing sub-system 202 (including one or more components on the integrated circuit 202), the one or more sensors 204, the controller 206, and the like.

The power source 214 may include any type of power source. For example, the power source 214 may include a rechargeable power source 214 such as, but not limited to, one or more rechargeable batteries, and the like.

In embodiments, the power source 214 may be powered by one or more solar panels 216. For example, where the power source 214 includes one or more rechargeable batteries, the one or more solar panels 216 may be used to recharge the one or more rechargeable batteries. The one or more solar panels 216 may be coupled to one or more external surfaces of the housing 200. For example, the one or more solar panels 216 may be coupled to one or more external surfaces of the top wall 201a, such that the one or more solar panels 216 are arranged to receive light from the sun.

Although FIGS. 2B-2C depict the housing 200 including the one or more solar panels 216, it is contemplated herein that the power source 214 may be charged using any charging techniques.

In embodiments, the monitoring device 102 includes a device level 218. For example, the monitoring device 102 may include a counterweight level 218 configured to adjust a direction of the monitoring device 102 within the liquid body. For instance, the counterweight level 218 may be configured to adjust the direction of the monitoring device 102 to position the one or more liquid monitoring sensors 101, 204 downward and the communication device 212 up, such that the monitoring device 102 is able to accurately monitor the liquid body and wirelessly transmit respective data to the one or more platform servers 104.

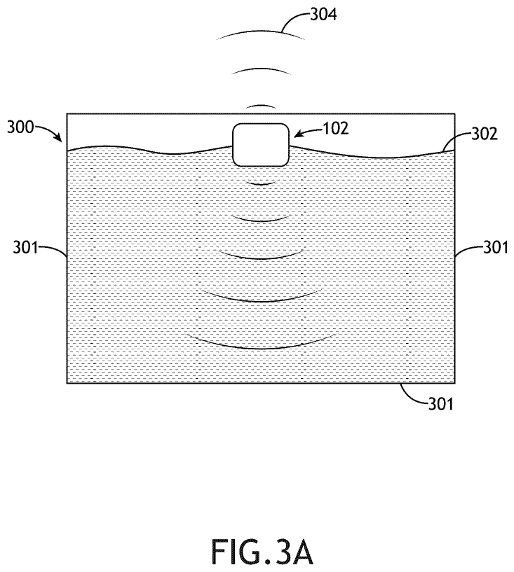

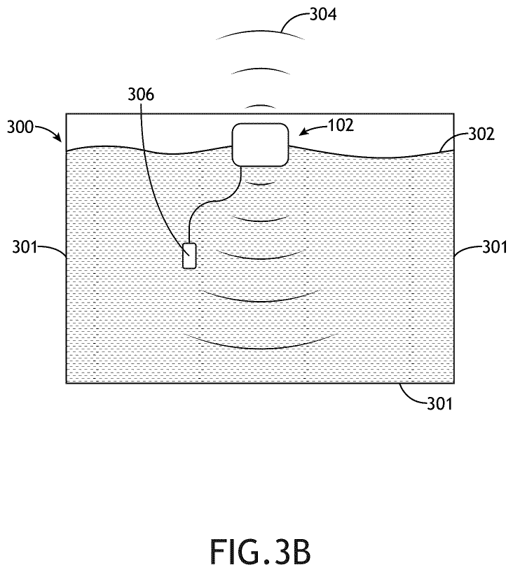

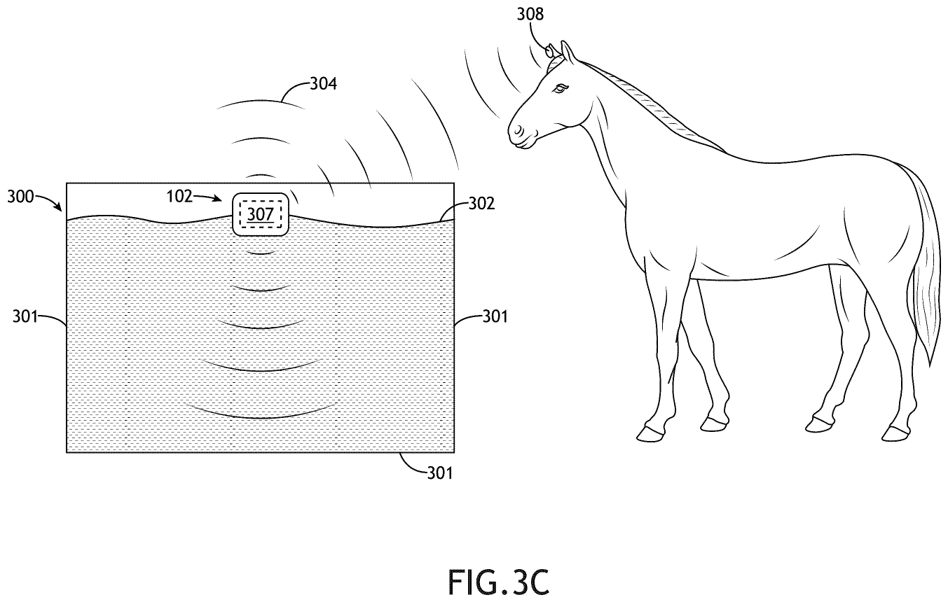

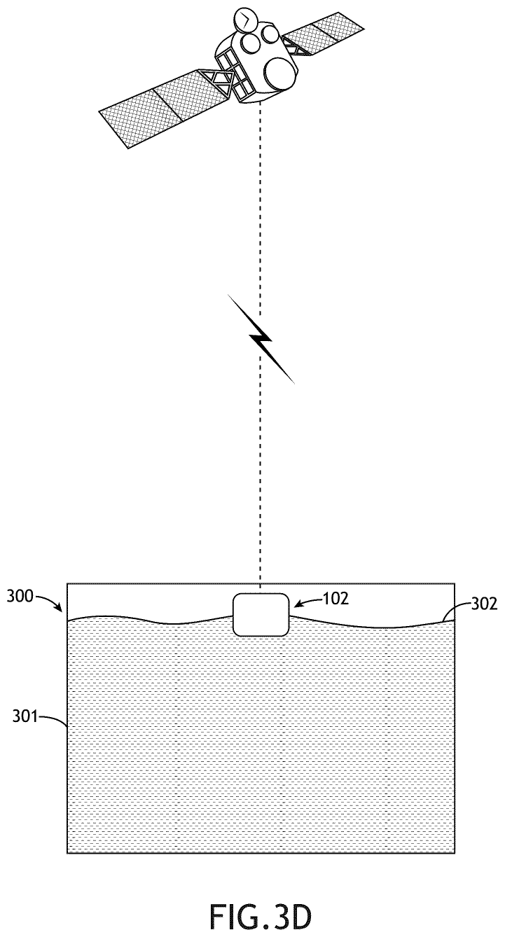

FIGS. 3A-3D are perspective views of the monitoring device 102 configured to collect and/or measure characteristics of a confined body of liquid 300, in accordance with one or more embodiments of the present disclosure. As previously discussed herein, the liquid monitoring system 100 may be configured to utilize non-contact sensing (i.e., no direct contact with the liquid during sensing) to determine one of the temperature, level, or quality of the liquid.

In embodiments, as shown in FIGS. 3A-3C, the monitoring device 102 may be configured for placement on a surface 302 of a liquid body 300. The liquid body 300 may be an at least partially confined body, where the confined body is at least partially defined by one or more confined space walls 301. Although FIGS. 3A-3C depict the confined body including four confined space walls 301, it is noted herein that the liquid body 300 may include any number of walls 301. For example, the liquid body 300 may be partially confined, such that the liquid body 300 includes three walls 301 (e.g., two sides walls and a bottom wall) with an opening.

In a non-limiting example, the liquid body 300 is a liquid storage tank (e.g., a water tank). In this example, the one or more confined space walls 301 may include one or more liquid storage tanks walls.

In another non-limiting example, the liquid body 300 is a pond (or another natural/artificial water source). In this example, the one or more confined space walls 301 may include one or more pond walls. In this regard, the confined space walls 301 may be one or more rocks, dirt, grass, or the like.

In another non-limiting example, the liquid body 300 is a fertigation or herbicide tank configured to hold one or more fertilizers or herbicides (or pesticides). In this example, the one or more confined space walls 301 may include one or more tank walls.

In another non-limiting example, the liquid body 300 is a swimming pool. In this example, the one or more confined space walls 301 may include one or more swimming pool walls.

In another non-limiting example, the liquid body 300 is a chemical tote. In this example, the one or more confined space walls 301 may include one or more chemical tote walls.

In embodiments, the monitoring device 102 may be configured to float on the surface 302 of the liquid body 300. For example, the monitoring device 102 may utilize a buoyant force to float on the surface 302 of the liquid body 300. In one instance, the monitoring device 102 may be dimensioned (e.g., shaped and/or sized) to utilize the buoyant force such the monitoring device 102 floats on the surface 302 of the liquid body 300. For example, as shown in FIG. 3A, the monitoring device 102 may have a conical shape, where the conical shape of the monitoring device 102 allows the monitoring device to float on the surface 302 of the liquid body 300.

By way of another example, the monitoring device 102 may fixed (or coupled) to a surface of the confined liquid body 300. For instance, the monitoring device 102 may be configured for installation on an existing structure located on or near the surface of the liquid body (e.g., a pump, a buoy, or the like). In this regard, the monitoring device 102 may be tethered to the liquid body 300.

In embodiments, as shown in FIG. 3A, the monitoring device 102 is configured to determine a level of the liquid body 300 based on interactions between one or more signals 304 and the confined space walls 301.

For example, the monitoring device 102 may be configured to generate one or more signals 304 using the one or more sensors 101, 204. In one instance, the monitoring device 102 generates one or more audible sound waves 304 using the one or more sensors 101, 204 (e.g., ultrasonic sensors, or the like), where the one or more audible sound waves 304 interact with the confined space walls 301 of the liquid body 300. In this regard, the interaction between the sound waves 304 and the confined space walls 301 may be analyzed to determine the level of the liquid body 300, where the liquid monitoring system 100 is configured to calculate a level of the liquid body 300 based on the time it takes for the sound waves 304 to return.

In another instance, the monitoring device 102 generates one or more laser pulses 304 using the one or more sensors 101, 204 (e.g., lidar sensors, or the like), where the one or more laser pulses 304 interact with the confined space walls 301 of the liquid body 300. In this regard, the interaction between the laser pulses 304 and the confined space walls 301 may be analyzed to determine the level of the liquid body 300, where the monitoring device 102 is configured to calculate a level of the liquid body 300 based on the time it takes for the one or more laser pulses 304 to bounce back.

In another instance, the monitoring device 102 generates one or more radar waves 304 using the one or more sensors 101, 204 (e.g., radar antennas, or the like), where the one or more radar waves 304 interact with the confined space walls 301 of the liquid body 300. In this regard, the interaction between the radar waves 304 and the confined space walls 301 may be analyzed to determine the level of the liquid body 300, where the monitoring device 102 is configured to calculate a level of the liquid body 300 based on the time it takes for the one or more radar waves 304 to bounce back.

In embodiments, the monitoring device 102 is configured to determine a level of the liquid body 300 based on a position or movement of the monitoring device 102. For example, the monitoring device 102 may include one or more acceleration sensors (e.g., accelerometers, gyroscopes, tilt switches, or the like) integrated within the device 102, where the one or more acceleration sensors may be configured to monitor changes in the device's position and movement relative to a surface of the liquid. In this regard, the liquid monitoring system 100 may then determine a level determination, in real-time, by capturing fluctuations in the device's height above the liquid, allowing for continuous tracking.

It is further contemplated herein the barometric pressure may be used to determine the liquid level (based on the height/position of the monitoring device 102). For example, the monitoring device 102 may include one or more barometric pressure sensors. However, barometric pressure, as with GPS (discussed further herein), varies based on atmospheric conditions, such that one or more reference points may be used to adjust for fluctuations. As such, a measurement point on the monitoring device 102 to determine a first barometric pressure and a reference point on the ground nearby (at a fixed height) to determine an additional barometric pressure, where the barometric pressures may be compared to account for atmospheric fluctuations. In this regard, fluctuations that are observed at the fixed reference point may be assumed to be due to atmospheric (or other) fluctuations. The measured height of the liquid surface may then be corrected by removing these fluctuations. Further, additional reference data such as, humidity data, may be used to adjust for fluctuations in barometric pressure based on atmospheric conditions.

It is contemplated herein that the shape of the monitoring device 102 may be such that when it is lying on a flat surface (such as on the bottom of a liquid storage tank), the liquid monitoring system 100 detects that the confined liquid body is empty.

In embodiments, as shown in FIG. 3A, the monitoring device 102 is configured to determine a temperature of the liquid body 300 based on interactions between one or more signals 304 and the liquid body 300. For example, the monitoring device 102 may be configured to generate one or more signals 304 using the one or more sensors 101, 204. In one instance, the monitoring device 102 generates one or more infrared waves 304 using the one or more sensors 101, 204 (e.g., infrared sensors, or the like), where the one or more infrared waves 304 interact with the liquid body 300. In this regard, the interaction between the infrared waves 304 and the liquid body 300 may be analyzed to determine the temperature of the liquid body 300, where the liquid monitoring system 100 is configured to calculate a temperature of the liquid body 300 based on the speed of the movement of the infrared waves 304.

In embodiments, as shown in FIG. 3B, the monitoring device 102 is coupled to one or more external direct measurement sensors 306. The one or more external sensors 306 may be configured to collect and/or measure additional data related to one or more characteristics of the liquid, where the one or more external sensors 306 directly interact with the liquid body. For example, the one or more external sensors 306 may be configured to integrate seamlessly into the device 102. By way of another example, the one or more external sensors 306 may be added as a separate component. In this regard, the capabilities of the liquid monitoring system 100 is expanded to allow for a broader range of data collection and analysis.

In embodiments, the one or more external sensors 306 includes one or more conductivity sensors configured to determine a conductivity of the liquid body 300. For example, the monitoring device 102 may configured to determine a temperature of the liquid body 300 based on conductivity of the liquid body 300 using the one or more external sensors 306, where the liquid monitoring system 100 is configured to calculate a temperature of the liquid body 300 based on the conductivity of the liquid body 300. In one instance, the liquid monitoring system 100 may be configured to utilize on-board reference tables (or look-up tables) to compare conductivity values and determine the temperature of the liquid. In another instance, the liquid monitoring system 100 may be configured to utilize an external database (or look-up table) to compare conductivity values and determine the temperature of the liquid.

By way of another example, the monitoring device 102 may configured to determine a relative quality (or cleanliness) of the liquid body 300 based on conductivity of the liquid body 300 using the one or more external sensors 306, where the liquid monitoring system 100 is configured to calculate a relative of the liquid body 300 based on the conductivity of the liquid body 300. In one instance, the liquid monitoring system 100 may be configured to utilize on-board reference tables (or look-up tables) to compare conductivity values and determine the relative cleanliness of the liquid. In another instance, the liquid monitoring system 100 may be configured to utilize an external database (or look-up table) to compare conductivity values and determine the relative cleanliness of the liquid.

It is contemplated that the liquid quality of the liquid body 300 may include any type of quality measurement. For example, the liquid quality measurement may be monitoring algae in liquid tanks (e.g., water tanks). By way of example, the liquid quality measurement may be monitoring nitrates in the liquid (e.g., water). By way of example, the liquid quality measurement may be monitoring chlorine in pools. By way of example, the liquid quality measurement may be monitoring toxic chemicals in the liquid (e.g., fish farm ponds, or the like).

In embodiments, the one or more external sensors 306 may be configured to interface with the liquid monitoring system 100 through wired or wireless means and accommodate various sensor requirements such as, but not limited to, analog, digital, or serial interfaces. In the wired configuration, the one or more external sensors 306 may be configured to communicate with the controller 206 of the monitoring device 102 via direct wiring. In the wireless configuration, the one or more external sensors 306 may be configured to communicate with the controller 206 of the monitoring device 102 via wireless means (e.g., Bluetooth, Wi-Fi, cellular (e.g., 3G, 4G, or 5G), or the like).

In embodiments, the one or more external sensors 306 may interface with the controller 206 of the monitoring device 102 while accommodating various sensor requirements. For example, the external sensor 306 may provide discrete analog measurements, such as 0-5V, 0-10V, 4-20 mA, or other compatible analog signals, depending on the specific sensor's capabilities. By way of another example, the external sensor 306 may employ digital or serial interfaces to communicate with the controller unit. It is contemplated here that the external direct measurement sensor 306 may help to expand the functionality of the liquid monitoring system 100 by capturing additional parameters such as, but not limited to, specific chemical concentrations, pH levels, or other liquid quality indicators, turbidity, salinity, flow rate, and the like. As such, the one or more external direct measurement sensors 306 may include any type of direct measurement sensor including, but not limited to, one or more pH sensors, one or more conductivity sensors, one or more turbidity sensors, one or more residual chlorine sensor, one or more dissolved oxygen sensors, one or more oxidation reduction potential sensors, one or more chemical oxygen demand sensors, one or more ammonia nitrogen sensors, one or more chlorophyll sensors, one or more algae sensors, one or more ion probe sensors, and the like.

It is noted herein that the integration of the one or more external direct measurement sensors 306 into the liquid monitoring system 100 may offer a range of improvements to the liquid monitoring system 100. For example, the external sensor 306 may eliminate the need to run sensors directly into the liquid tank, ensuring that they remain housed safely within the main monitoring package below the liquid surface. By way of another example, the external sensor 306 may prevent interference from external factors such as livestock, ensuring uninterrupted and reliable data collection. By way of another example, the external sensor 306 may be added or replaced with ease as new sensor solutions become available, keeping the monitoring infrastructure up-to-date with evolving industry standards. By way of another example, the external sensor 306 may be attached to associated equipment, such as pumps or wells, to provide additional monitoring capabilities and enabling users to gain insights into critical parameters (e.g., flow rates, activity, energy usage, or the like).

Although FIG. 3B depicts the external measurement sensors 306 within the liquid body 300, it is contemplate that the external measurement sensors 306 may be placed outside the liquid body 300. For example, the external measurement sensors 306 may be placed on a gate to know when it was opened or if it was closed. By way of another example, the external measurement sensors 306 may be placed on a pump or well to detect whether the pump/well is moving or running based on sensed vibration, etc.

In embodiments, as shown in FIG. 3C, the monitoring device 102 includes a radio frequency identification (RFID) device. For example, the RFID reading device 307 may be integrated into the monitoring device 102 (or added as a separate component). For instance, the RFID reading device 307 may be configured to communicate with one or more RFID tags 308 via one or more RFID signals. In a non-limiting example, the RFID reading device 307 may be configured to track and manage livestock effectively within a vicinity of the liquid body 300, where the one or more RFID tags 308 are affixed to animals and read by the RFID reading device 307 as the animals come into proximity with the liquid monitoring system 100. It is noted that this enables livestock tracking and identification, offering insights into individual animal behavior (e.g., frequency of watering), movement patterns, and overall health of the animals.

Although the above discussion is directed to affixing RFID tags 308 to animals (e.g., livestock), it is noted that the RFID reading device 307 may be used to monitor people, vehicles, equipment, or the like. For example, the RFID reading device 307 may be used with mobile devices that emit RFID, people wearing RFID tags, or vehicles or equipment including RFID tags. Therefore, the above discussion should not be construed as limiting the scope of the present disclosure.

It is contemplated here in that the RFID reading device 307 may operate in an active mode (where the RFID tag 308 actively transmits a signal and the RFID reading device 307 receives the signal) or a passive mode (where the RFID reading device 307 transmits an interrogation signal, which is received by the RFID tag 308 and serves to activate the RFID tag 308). Further, it is completed herein that the monitoring device 102 may include any type of passive or active wireless communication device suitable for monitoring livestock management and is not limited to an RFID device.

It is contemplated herein that the liquid monitoring system 100 may allow for real-time assessment of animal well-being by continuously monitoring their presence and behavior. The liquid monitoring system 100 may be configured to provide an alert or notification based on RFID tag readings, aiding in the early detection of issues such as animal distress or anomalies in feeding patterns. The integration of an RFID reading device 307 into the liquid monitoring system 100 not only benefits livestock management but also offers valuable insights for agricultural operations. By correlating data from the RFID tags with liquid level measurements, farmers and ranchers may gain a holistic view of how environmental factors, such as water availability, impact the animals' behavior and their overall productivity.

Further, it is contemplated herein that the integration of an RFID reading device 307 into the liquid monitoring system 100 may offer a variety of advantages. For example, RFID technology may enable the capture of livestock within the range of the drinking area, which allows for the identification and tracking of potentially at-risk animals within a herd. By way of another example, the RFID reading device may contribute to the liquid monitoring system 100's ability to conduct herd audits in real-time, maintaining an accurate count of animals. By way of another example, the RFID reading device 307 may provide insights into the drinking habits and activity patterns of the animals, aiding in the optimization of feeding and watering schedules. By way of another example, notifications triggered by RFID tag readings may prompt caretakers to approach specific animals and address their unique needs promptly. Furthermore, the RFID reading device 307 may be capable of integrating with other connected devices and data streams.

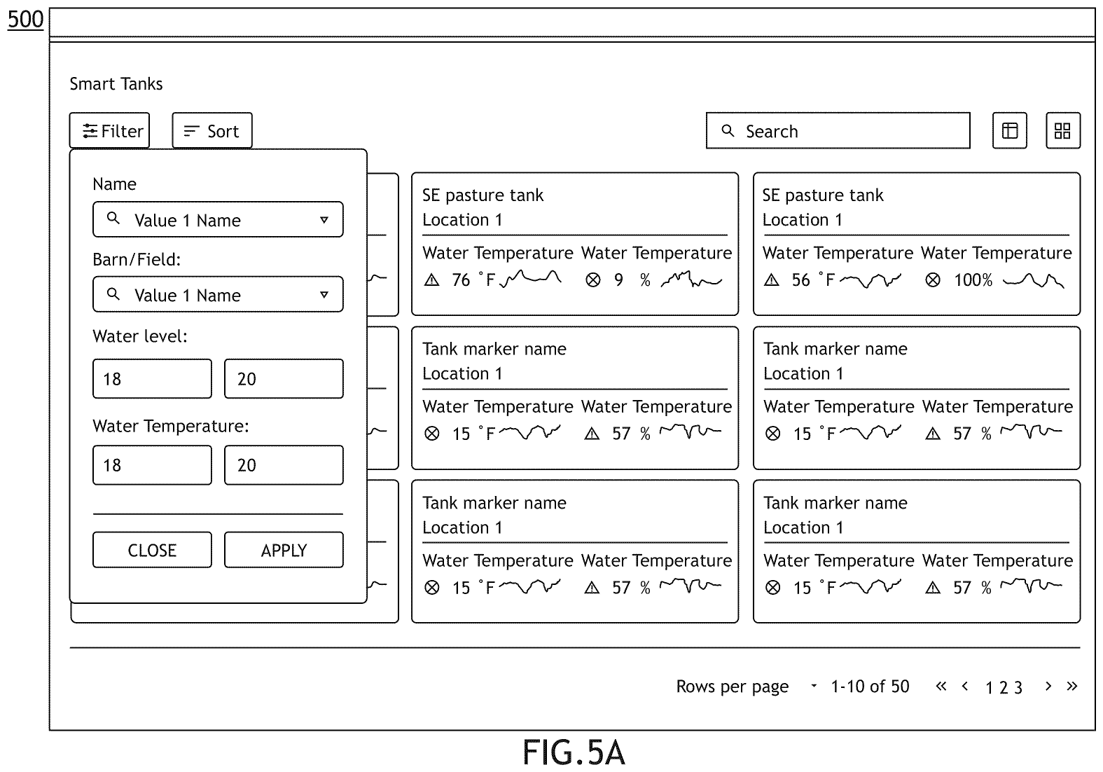

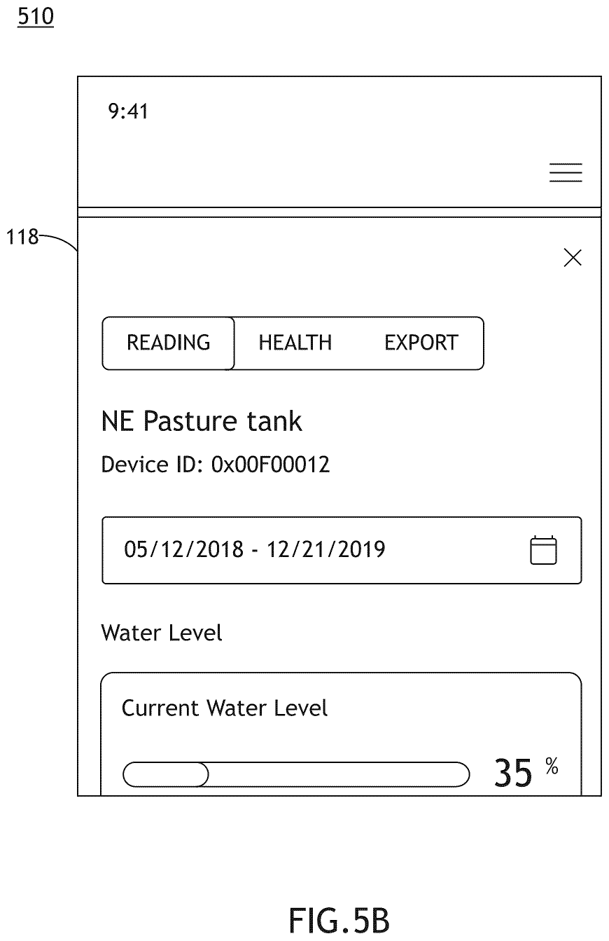

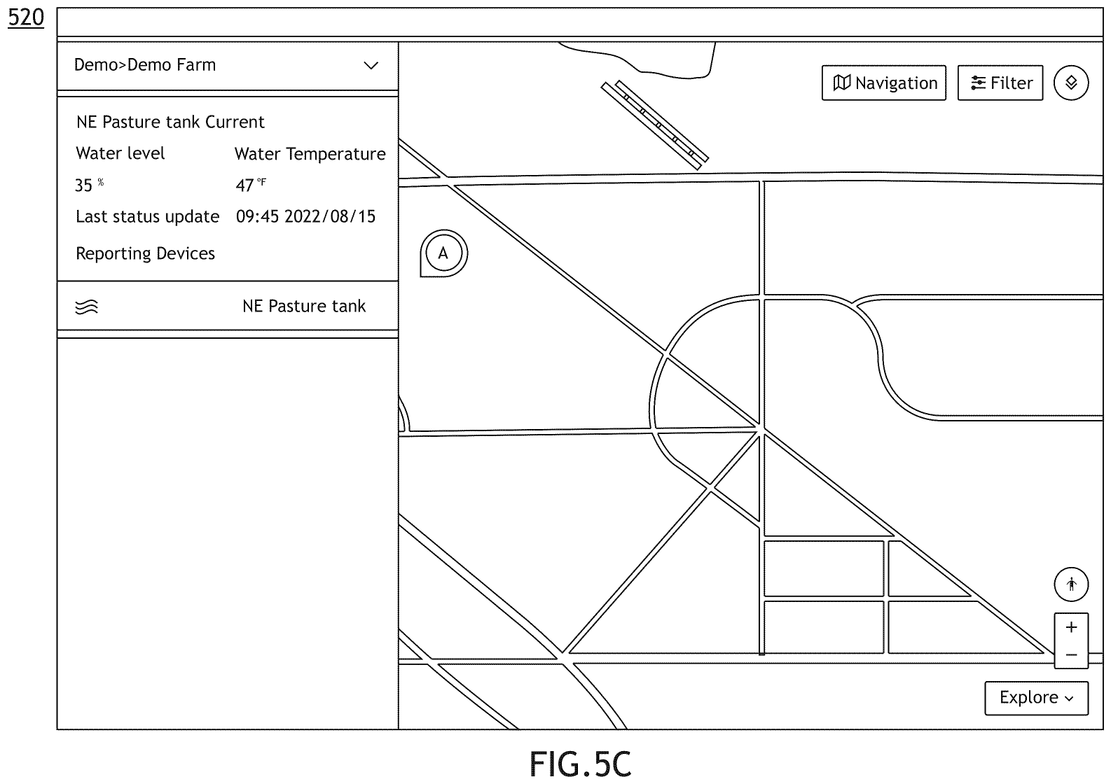

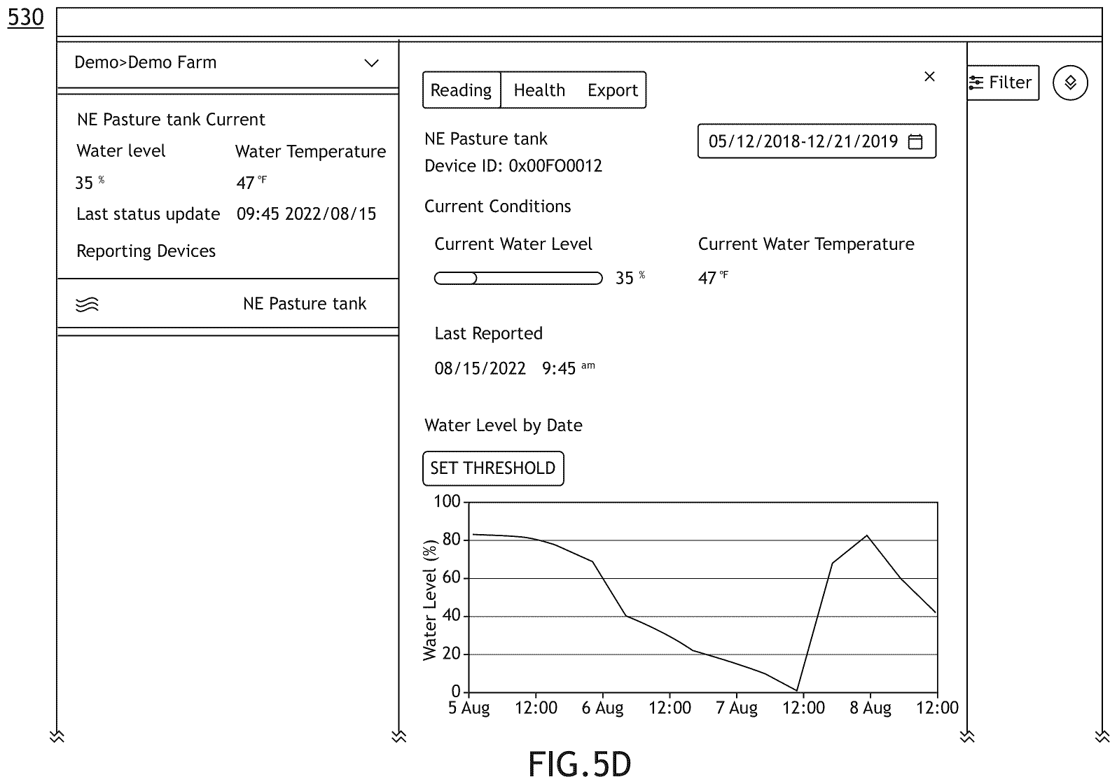

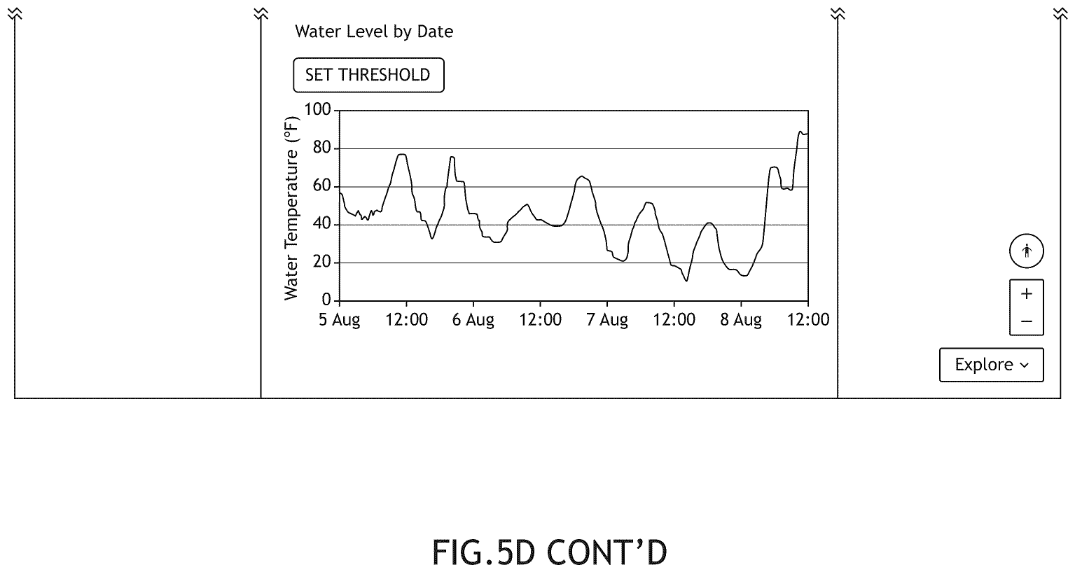

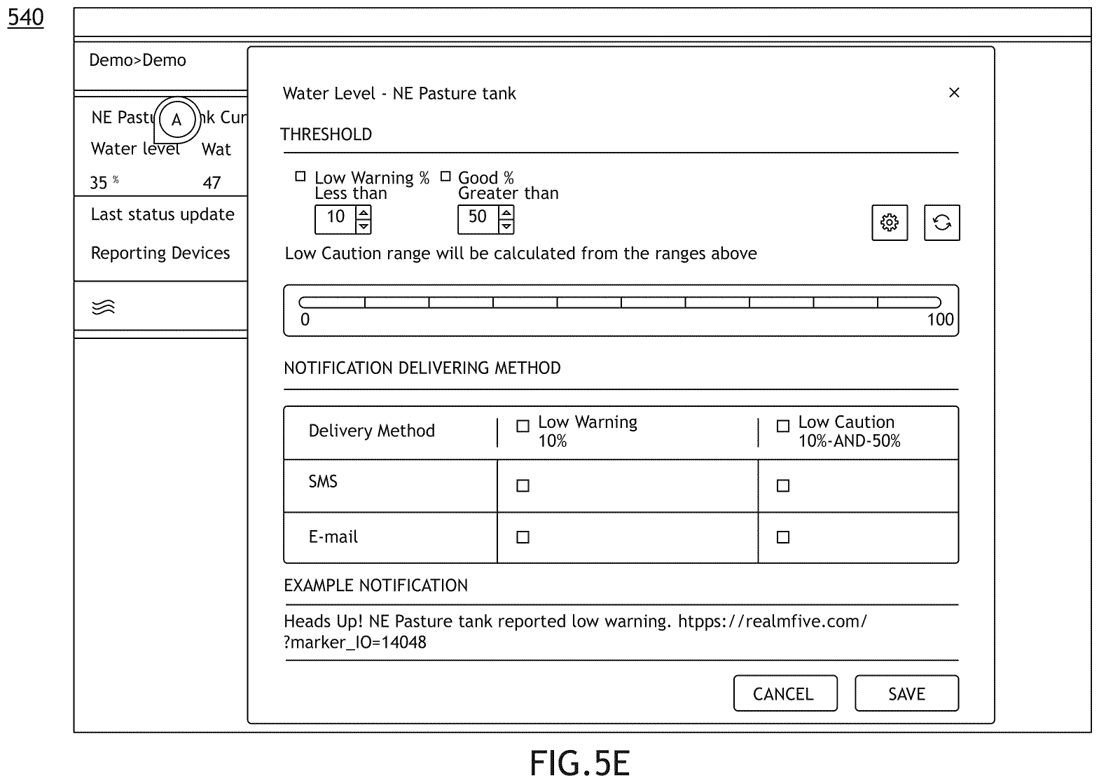

In embodiments, as shown in FIG. 5D, the liquid monitoring system 100 may utilize real-time kinetic (RTK) techniques (or post processed kinematics) to monitor the liquid. For example, the monitoring device 102 may include one or more rovers (including a rover global positioning system (GPS) unit), where a base (including a GPS unit) may be installed at a location adjacent to the liquid body. In this regard, positioning data from the GPS units may be used to monitor the liquid level.

It is contemplated herein that GPS readings may fluctuate based on, for example, atmospheric interference. As such, one or more reference points may be used to adjust for the fluctuations. For example, the one or more reference points may include a reference point on the monitoring device 102 to measure variable height (as the monitoring device 102 floats up or down) and a reference point on the ground nearby (at a known height), where the reference points may be compared to account for atmospheric interference fluctuations. In this regard, fluctuations that are observed at the fixed altitude reference point may be assumed to be due to atmospheric (or other) fluctuations. The measured height of the liquid surface may then be corrected by removing these fluctuations.

The base may be communicatively coupled to the one or more rovers. For example, one or more base communication circuits may be configured to communicate with one or more rover communication circuits. The one or more base communication circuits may be configured to transmit and/or receive data to the one or more rovers using any type of wireless communication technique known in the art including, but not limited to, radio frequency (RF) protocols, Bluetooth protocols, GSM, GPRS, DCMA, EV-DO, EDGE, WIMAX, 4G, 4G LTE, 5G, 6G, WiFi protocols, RF, LoRa, and the like.

Liquid level monitoring using real-time kinetic (RTK) positioning techniques is generally discussed in U.S. Patent Publication Number 2021/0025747, published on Jan. 28, 2021, which is incorporated herein by reference in the entirety.

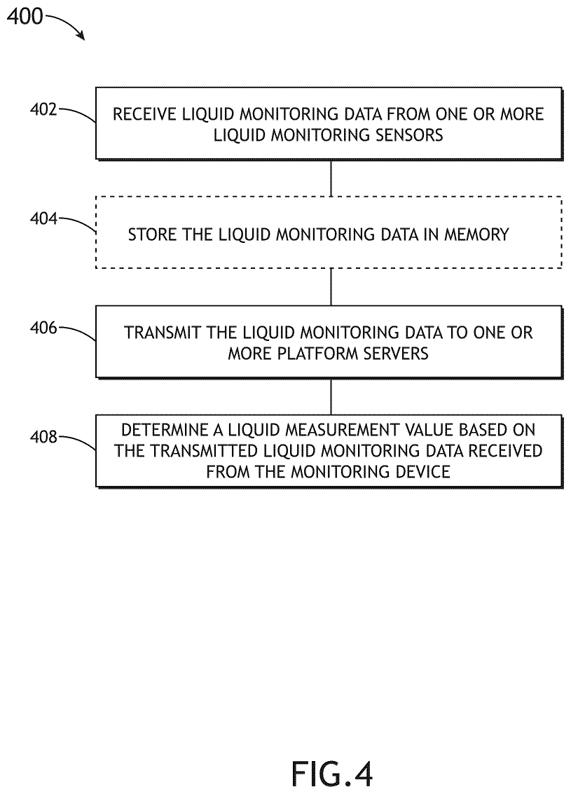

FIG. 4 illustrates a process flow diagram illustrating the steps of a method 400 for liquid monitoring, in accordance with one or more embodiments of the present disclosure. It is noted herein that the step of method 400 may be implemented all or in part by liquid monitoring system 100. It is further recognized, however, that the method 400 is not limited to the liquid monitoring system 100 in that additional or alternative system-level embodiments may carry out all or part of the steps of method 400.

In a step 402, liquid monitoring data may be received from the one or more liquid monitoring sensors. For example, the one or more liquid monitoring sensors 101, 204 of the monitoring device 102 may be configured to measure and/or collect data related to one or more characteristics of the liquid and provide the data to the controller 206 of the monitoring device 102. For instance, the liquid monitoring data may include, but is not limited to, liquid level data, liquid temperature data, or liquid quality data;

In an optional step 404, the liquid monitoring data may be stored in memory. For example, the controller 206 of the monitoring device 102 may be configured to store the liquid monitoring data in memory 210 on the monitoring device 102.

In a step 406, the liquid monitoring data may be provided to the one or more platform servers. For example, the controller 206 of the monitoring device 102 may be configured to transmit the data to the one or more platform servers 104. In one instance, as shown in FIG. 1A, the monitoring device 102 may be configured to directly transmit the data to the one or more platform servers 104 via the network 110. In another instance, as shown in FIG. 1B, the monitoring device 102 may be configured to transmit the data to the one or more platform servers 104 via the data gateway 114, where the data gateway 114 transmits the data to the platform servers 104 via the network 110.

In a step 408, one or more liquid measurement values may be determined based on the transmitted liquid monitoring data received from the monitoring device. The one or more liquid measurement values may include, but are not limited to, liquid level, temperature, cleanliness (or quality), or the like. For example, the one or more processors may be configured to determine one or more liquid measurement values based on the transmitted liquid monitoring data received from the monitoring device 102. In one instance, the one or more processors 106 of the one or more platform servers 104 may be configured to determine one or more liquid measurement values based on the transmitted liquid monitoring data received from the monitoring device 102. In another instance, the one or more processors 208 of the controller 206 of the monitoring device 102 may be configured to determine one or more liquid measurement values based on the transmitted liquid monitoring data received from the monitoring device 102.

In embodiments, determined liquid level (or depth) may be reported as a raw measurement (e.g., millimeters, inches, or the like) or correlated to a percentage. For example, where the measurements of the liquid body at full capacity are given, the level may be reported as a percent full based on the given full capacity.

In embodiments, the determined liquid temperature may be reported as a raw measurement (e.g., ° C., ° F., K).

It is contemplated herein that where connectivity is poor, the monitoring device 102 may include a read-out device (or screen) for a user to monitor determined liquid measurement values on-site. In this non-limiting example, the monitoring device 102 may utilize one or more on-board databases/tables to determine the one or more liquid measurement values and display such results on the read-out device, such that such measurements may be viewed/displayed on-site.

In embodiments, the system 100 is configured to determine a liquid health based on at least one of the determined liquid level, temperature, or quality. For example, the one or more processors may be configured to determine a liquid health of the confined liquid body based on the determined one or more liquid measurement values. In one instance, the one or more processors 106 of the one or more platform servers 104 may be configured to determine a liquid health of the confined liquid body based on the determined one or more liquid measurement values. In another instance, the one or more processors 208 of the controller 206 of the monitoring device 102 may be configured to determine a liquid health of the confined liquid body based on the determined one or more liquid measurement values.

In embodiments, the system 100 may be configured to dispense one or more chemicals into the configured liquid body 300 based on the determined one or more liquid measurements. For example, the monitoring device 102 may include (or be coupled to) a dispensing device configured to dispense one or more liquids into the confined liquid body. In one instance, the dispensing device may be configured to dispense copper sulfate into the liquid body 300 to prevent growth of algae in the liquid storage tank. In another instance, the dispensing device may be configured to dispense chlorine into the liquid body 300 (e.g., swimming pool).

In a step 410, one or more signals may be generated to cause a display of a user device to display the determined one or more liquid measurement values on a graphic user interface (GUI) to a user. For example, the one or more processors 106 may be configured to generate the one or more signals to cause the display 118 of the user device 116 to display the GUI to a user, where the GUI displays the determined one or more liquid measurement values.

It is contemplated herein that a user (or operator) may desire to view liquid measurement values (e.g., temperatures, levels, quality, etc.) remotely via the user device 116. In this regard, the liquid monitoring system 100 may provide the ability to view liquid characteristics (e.g., temperature, liquid levels, electroconductivity levels, and the like) at various liquid bodies throughout a farm/pasture, such that the farm owner or operator may adjust one or more farm characteristics or farming operating parameters in order to more effectively and efficiently operate the farm. For instance, based on liquid levels, temperature, quality, etc. collected, stored, and displayed to a farm owner via display 118, a farm owner may be able refill the liquid within the tank, replace the liquid within the tank, and the like based on the data (e.g., refill rate, empty rate, etc.).

In embodiments, as shown in FIGS. 5A-5B, the display 118 of user device 116 may display a GUI 500, 510 depicting the one or more liquid measurement values for one or more liquid bodies (e.g., water tanks). For example, as shown in FIG. 5A, the GUI 500 may display one or more water temperatures for a plurality of liquid bodies (e.g., water tanks) in a first view (e.g., a desktop view). By way of another example, as shown in FIG. 5B, the GUI 510 may display one or more water temperatures for a plurality of liquid bodies (e.g., water tanks) in a second view (e.g., mobile view) using a mobile device.

In embodiments, as shown in FIG. 5C, the user device 116 may display a GUI 520 depicting a map. For example, GUI 520 may display one or more locations of one or more liquid bodies, such that the operator may select one or more liquid bodies to monitor. In this regard, the GUI 520 may display the water level and water temperate for a selected one or more liquid bodies (e.g., water tanks).

In embodiments, as shown in FIG. 5D, the user device 116 may display a GUI 530 depicting the one or more liquid measurement values over time. For example, GUI 530 may display one or more graphs (or plots) showing the measurement value trend for a selected liquid body (e.g., water tank). For instance, as shown in FIG. 5D, the GUI 530 may display a water level by date plot and a water temperature by date plot, showing the respective values over a period of time (e.g., hours, days, weeks, months, years, or the like).

In a step 412, one or more alert signals may be generated to cause a user device to generate one or more alerts for a user. For example, the one or more alert signals may be generated based on one or more predetermined thresholds. For instance, one or more thresholds may be adjusted (or set) using the user device 116. In embodiments, as shown in FIG. 5E, one or more thresholds may be adjusted using the GUI 540.

In embodiments, the liquid monitoring system 100 is configured to transmit one or more alerts to a user. For example, the one or more processors may be configured to transmit one or more alerts to a user, where the user device 116 is configured to display the one or more alerts to the user via the display 118. For example, the one or more processors may be configured to transmit one or more alerts to a user indicating a low liquid level. By way of another example, the one or more processors may be configured to transmit one or more alerts to a user indicating a low/high liquid temperature. By way of another example, the one or more processors may be configured to transmit one or more alerts to a user indicating a poor liquid quality. By way of another example, the one or more processors may be configured to transmit one or more alerts to a user indicating a leak is detected (e.g., where the tank is emptying faster than defined and monitored). By way of another example, the one or more processors may be configured to transmit one or more alerts to a user indicating an overfill if the water level threshold is exceeded.

The one or more alerts generated by liquid monitoring system 100 may include any alert known in the art including, but not limited to, aural alerts (or sound alerts), automated phone calls, text messages, emails, application notifications, banners, push notifications, and the like.

It is contemplated herein that one or more steps of the method 400 may be carried out by any of the processors of the liquid monitoring system 100. Therefore, the above description shall not be construed as limiting the scope of the present disclosure. For example, the one or more processors 208 of the controller 206 of the monitoring device 102 may be configured to determine the one or more liquid measurement values (in step 408) and provide the determined one or more liquid measurement values to the one or more platform servers 104.

Referring again to FIGS. 1A-1B, in embodiments, the one or more processors 106, 208 may include any one or more processing elements known in the art. In this sense, the one or more processors 106, 208 may include any microprocessor-type device configured to execute software algorithms and/or instructions. For example, the one or more processors 106, 208 may consist of a desktop computer, mainframe computer system, workstation, image computer, parallel processor, or other computer system (e.g., networked computer) configured to execute a program configured to operate the system 100, as described throughout the present disclosure. It should be recognized that the steps described throughout the present disclosure may be carried out by a single computer system or, alternatively, multiple computer systems. Furthermore, it should be recognized that the steps described throughout the present disclosure may be carried out on any one or more of the one or more processors 106, 208. In general, the term “processor” may be broadly defined to encompass any device having one or more processing elements, which execute program instructions from memory 108, 210. Moreover, different subsystems of the system 100 (e.g., user device 116, network 110, server 104) may include processor or logic elements suitable for carrying out at least a portion of the steps described throughout the present disclosure. Therefore, the above description should not be interpreted as a limitation on the present disclosure but merely an illustration.

The memory 108, 210 may include any storage medium known in the art suitable for storing program instructions executable by the associated one or more processors 106. For example, the memory 108, 210 may include a non-transitory memory medium. For instance, the memory 108, 210 may include, but is not limited to, a read-only memory (ROM), a random-access memory (RAM), a magnetic or optical memory device (e.g., disk), a solid-state drive, and the like. It is further noted that memory 108, 210 may be housed in a common controller housing with the one or more processors 106, 208. In an alternative embodiment, the memory 108, 210 may be located remotely with respect to the physical location of the processors 106, 208, user device 116, server 104, and the like. For instance, the one or more processors 106, 208 and/or the server 104 may access a remote memory (e.g., server), accessible through a network (e.g., internet, intranet and the like). The memory 108, 210 may also maintain program instructions for causing the one or more processors 106, 208 to carry out the various steps described through the present disclosure.

Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

All of the methods described herein may include storing results of one or more steps of the method embodiments in memory. The results may include any of the results described herein and may be stored in any manner known in the art. The memory may include any memory described herein or any other suitable storage medium known in the art. After the results have been stored, the results can be accessed in the memory and used by any of the method or system embodiments described herein, formatted for display to a user, used by another software module, method, or system, and the like. Furthermore, the results may be stored “permanently,” “semi-permanently,” temporarily,” or for some period of time. For example, the memory may be random access memory (RAM), and the results may not necessarily persist indefinitely in the memory.

It is further contemplated that each of the embodiments of the method described above may include any other step(s) of any other method(s) described herein. In addition, each of the embodiments of the method described above may be performed by any of the systems described herein.

One skilled in the art will recognize that the herein described components operations, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected,” or “coupled,” to each other to achieve the desired functionality, and any two components capable of being so associated can also be viewed as being “couplable,” to each other to achieve the desired functionality. Specific examples of couplable include but are not limited to physically mateable and/or physically interacting components and/or wirelessly interactable and/or wirelessly interacting components and/or logically interacting and/or logically interactable components.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. A liquid monitoring system, the liquid monitoring system comprising:
a monitoring device, the monitoring device configured to be positioned at a surface of a confined liquid body, the monitoring device comprising:
a housing including one or more walls at least partially defining a cavity; and
an internal sensing sub-system, the cavity of the housing configured to enclose the internal sensing sub-system, the internal sensing sub-system comprising one or more liquid monitoring sensors, the one or more liquid monitoring sensors configured to monitor at least one of a level, a temperature, or a quality of the confined liquid body; and
one or more processors communicatively coupled to the one or more liquid monitoring sensors, the one or more processors configured to execute a set of program instructions in a memory, the set of program instructions configured to cause the one or more processors to:
receive a set of liquid monitoring data from the one or more liquid monitoring sensors, the set of liquid monitoring data including at least one of liquid level data, liquid temperature data, or liquid quality data for the confined liquid body; and
determine one or more liquid measurement values based on the set of liquid monitoring data, the determined one or more liquid measurement values including at least one of a liquid level measurement value, a temperature measurement value, or a liquid quality measurement value for the confined liquid body.

2. The system of claim 1, wherein the one or more liquid monitoring sensors having no direct contact with the confined liquid body.

3. The system of claim 1, wherein the monitoring device has a conical shape.

4. The system of claim 1, wherein the one or more liquid monitoring sensors include one or more ultrasonic sensors configured to generate one or more ultrasonic sound waves.

5. The system of claim 4, wherein determine one or more liquid measurement values based on the set of liquid monitoring data comprises:
determining the liquid level measurement value based on the ultrasonic sound waves.

6. The system of claim 1, wherein the one or more liquid monitoring sensors include one or more lidar sensors configured to generate one or more laser pulses.

7. The system of claim 6, wherein determine one or more liquid measurement values based on the set of liquid monitoring data comprises:
determining the liquid level measurement value based on the one or more laser pulses.

8. The system of claim 1, wherein the one or more liquid monitoring sensors include one or more radar antennas configured to generate one or more radar waves.

9. The system of claim 8, wherein determine one or more liquid measurement values based on the set of liquid monitoring data comprises:
determining the liquid level measurement value based on the one or more radar waves.

10. The system of claim 1, wherein the one or more liquid monitoring sensors include one or more acceleration sensors configured to determine a position of the monitoring device.

11. The system of claim 10, wherein determine one or more liquid measurement values based on the set of liquid monitoring data comprises:
determining the liquid level measurement value based on the position of the monitoring device.

12. The system of claim 1, wherein the one or more liquid monitoring sensors include one or more infrared sensors configured to generate infrared radiation.

13. The system of claim 12, wherein determine one or more liquid measurement values based on the set of liquid monitoring data comprises:
determining the liquid temperature measurement value based on the infrared radiation.

14. The system of claim 1, wherein the one or more liquid monitoring sensors include one or more conductivity sensors configured to determine a conductivity of the confined liquid body.

15. The system of claim 14, wherein determine one or more liquid measurement values based on set of liquid monitoring data comprises:
determining the liquid temperature measurement value based on the conductivity of the confined liquid body and reference conductivity data.

16. The system of claim 15, wherein the reference conductivity data includes data stored in a remote database or an on-board table.

17. The system of claim 14, wherein determine one or more liquid measurement values based on the set of liquid monitoring data comprises:
determining the liquid quality measurement value based on the conductivity of the confined liquid body and reference conductivity data.

18. The system of claim 17, wherein the reference conductivity data includes data stored in a remote database or an on-board table.

19. The system of claim 1, further comprising:
one or more power sources.

20. The system of claim 19, further comprising:
one or more solar panels coupled to one or more external surfaces of the housing, the one or more solar panels configured to provide power to the one or more power sources.

21. The system of claim 1, wherein the internal sensing sub-system includes one or more integrated circuits, wherein the one or more liquid monitoring sensors are coupled to one or more surfaces of the one or more integrated circuits.

22. The system of claim 1, wherein the monitoring device further comprises:
one or more additional liquid monitoring sensors arranged within the cavity of the housing.

23. The system of claim 22, wherein the one or more additional liquid monitoring sensors do not make direct contact with the confined liquid body.

24. The system of claim 22, wherein the one or more additional liquid monitoring sensors include a temperature sensing probe.

25. The system of claim 22, wherein the monitoring device further comprises:
a counterweight level configured to adjust a direction of the liquid monitoring system to position the one or more additional liquid monitoring sensors downward.

26. The system of claim 1, wherein the monitoring device further comprises:
a controller including one or more processors communicatively coupled to the one or more liquid monitoring sensors, the one or more processors configured to execute a set of program instructions in a memory; and
one or more communication devices.

27. The system of claim 26, further comprising:
one or more platform servers communicatively coupled to the controller of the monitoring device via a network,
the one or more platform servers comprising one or more processors and one or more communication devices, the one or more processors configured to execute a set of program instructions in a memory.

28. The system of claim 27, further comprising:
a data gateway, wherein the data gateway is communicatively coupled to one or more platform servers via the network.

29. The system of claim 1, wherein the set of program instructions are further configured to cause the one or more processors to:
generate one or more signals configured to cause a display of a user device to display a graphical user interface depicting the determined one or more liquid measurement values.

30. The system of claim 1, wherein the set of program instructions are further configured to cause the one or more processors to:
generate one or more alerts based on one or more predetermined thresholds and the determined one or more liquid measurement values.

31. The system of claim 30, wherein the generated one or more alerts comprise:
one or more aural alerts or one or more visual alerts.

32. The system of claim 1, further comprising:
one or more direct measurement sensors communicatively coupled to the monitoring device, wherein the one or more direct measurement sensors make direct contact with the confined liquid body.

33. The system of claim 32, wherein the one or more direct measurement sensors comprise at least one of:
one or more pH sensors, one or more conductivity sensors, one or more turbidity sensors, one or more residual chlorine sensor, one or more dissolved oxygen sensors, one or more oxidation reduction potential sensors, one or more chemical oxygen demand sensors, one or more ammonia nitrogen sensors, one or more chlorophyll sensors, one or more algae sensors, or one or more ion probe sensors.

34. The system of claim 1, wherein the monitoring device further comprises:
one or more radio frequency identification (RFID) readers configured to communicate with one or more RFID tags positioned external to the housing.

35. The system of claim 34, wherein the one or more RFID tags are affixed to a livestock animal, wherein the one or more RFID readers are configured to detect when the livestock animal comes within a defined region of the confined liquid body.

36. The system of claim 1, wherein the confined liquid body includes water within a water storage tank.

37. The system of claim 1, wherein the confined liquid body includes herbicides within an herbicide tank.

38. The system of claim 1, wherein the confined liquid body includes fertilizers within fertigation tank.

39. A liquid monitoring device, the liquid monitoring device comprising:
a housing including one or more walls at least partially defining a cavity; and
an internal sensing sub-system, the cavity of the housing configured to enclose the internal sensing sub-system, the internal sensing sub-system comprising one or more liquid monitoring sensors, the one or more liquid monitoring sensors configured to monitor at least one of a level, a temperature, or a quality of a confined liquid body,
the monitoring device configured to be positioned at a surface of the confined liquid body.

40. The device of claim 39, wherein the one or more liquid monitoring sensors have no direct contact with the confined liquid body.

41. The device of claim 39, wherein the monitoring device has a conical shape.

42. The device of claim 39, wherein the monitoring device further comprises:
one or more additional liquid monitoring sensors arranged within the cavity of the housing.

43. The device of claim 42, wherein the one or more additional liquid monitoring sensors do not make direct contact with the confined liquid body.

44. The device of claim 42, wherein the one or more additional liquid monitoring sensors include a temperature sensing probe.

45. The device of claim 39, wherein the monitoring device further comprises:
a counterweight level configured to adjust a direction of the monitoring device to position one of the one or more liquid monitoring sensors or one or more additional liquid monitoring sensors downward.

46. The device of claim 39, further comprising:
one or more power sources.

47. The device of claim 46, further comprising:
one or more solar panels coupled to one or more external surfaces of the housing, the one or more solar panels configured to provide power to the one or more power sources.

48. The device of claim 39, wherein the internal sensing sub-system includes one or more integrated circuits, wherein the one or more liquid monitoring sensors are coupled to one or more surfaces of the one or more integrated circuits.

49. A method comprising:
receiving a set of liquid monitoring data from one or more liquid monitoring sensors, the one or more liquid monitoring sensors having no direct contact with a confined liquid body, the set of liquid monitoring data including at least one of liquid level data, liquid temperature data, or liquid quality data; and
determining one or more liquid measurement values based on the set of liquid monitoring data, the determined one or more liquid measurement values including at least one of a liquid level measurement value, a temperature measurement value, or a liquid quality measurement value for the confined liquid body.
